(19) United States
(12) Patent Application Publication
(10) Pub. No.: US 2022/0346350 A1
(43) Pub. Date: Nov. 3, 2022

(54) DUST SUCTION CABIN AND NAIL GRINDER SET

(71) Applicant: HAPPY UNITOYS CO., LIMITED, Shenzhen (CN)

(72) Inventor: Qinsheng Li, SHENZHEN (CN)

(21) Appl. No.: 17/329,339

(22) Filed: May 25, 2021

(30) Foreign Application Priority Data
Apr. 29, 2021 (CN) 202110472728.4

(51) Int. Cl. A01K 13/00; A45D 29/05

(52) U.S. Cl. CPC A01K 13/00; A45D 29/05

(57) ABSTRACT

Provided is a dust suction cabin and a nail grinder set. The dust suction cabin includes a cabin body and a dust suction device. The cabin body defines a nail grinding chamber that is used for grinding the nail of a pet, and a chamber wall of the nail grinding chamber is at least partially transparent. The cabin body further defines a dust removal port and two inlets communicating with the nail grinding chamber. One of the two inlets is used for the nail grinder to enter the nail grinding chamber, and the other is used for the pet's claw to extend into the nail grinding chamber. The dust suction device is used for sucking out the scraps generated during the pet nail grinding process through the dust removal port.

### CROSS-REFERENCE TO RELATED APPLICATIONS

This application claims the benefit and priority of China patent application No. 202110472728.4 filed on Apr. 29, 2021, disclosure of which is hereby incorporated by reference in its entirety.

### TECHNICAL FIELD

This disclosure relates to the technical field of pet nail grinding, and more particularly relates to a dust suction cabin and a nail grinder set.

### BACKGROUND

In order to get rid of loneliness or pursue entertainment, people start to keep pets for accompany. Cats and dogs are often popular for their cuteness and loyalty. However, cats and dogs grow nails just like humans. Pointed nails can easily scratch the pet owner or damage the furniture, so the pet owner needs to trim the pet's nails regularly.

The related art pet nail grinders tend to emit unpleasant odors when grinding pet nails, nor can they collect the scraps produced during the process of grinding the pet's nails. The scraps produced are scattered around and are likely to remain on the pet owner's clothes or be inhaled by the pet owner, so that the bacteria on the scraps are easy to parasitize in or on the pet owner's body, affecting the pet owner's physical and mental health.

At present, when a pet owner uses a pet nail grinder to grind his/her pet's nails, he/she may easily grind to the blood-lined part of the pet's nail due to factors such as low ambient light, the inability to observe the grinded part of the nail at close range during grinding, etc., thus causing injury to the pet.

### SUMMARY

It is therefore a main objective of the present disclosure to provide a dust suction cabin, which aims to solve the technical problem that the related art pet nail grinders cannot guarantee the sanitation of the operating environment when performing nail grinding operations.

In order to achieve the above objective, the dust suction cabin proposed by the present disclosure includes a cabin body and a dust suction device. The cabin body defines a nail grinding chamber that is used for grinding the nail of a pet, and a chamber wall of the nail grinding chamber is at least partially transparent. The cabin body further defines a dust removal port and two inlets communicating with the nail grinding chamber. One of the two inlets is used for the nail grinder to enter the nail grinding chamber, and the other is used for the pet's claw to extend into the nail grinding chamber. The dust suction device is used for sucking out the scraps generated during the pet nail grinding process through the dust removal port.

In some embodiments, the dust suction cabin further includes two support arms, which are respectively arranged at the two inlets and extend from the inlets in a direction away from the cabin body. The support arms each define a support channel, where one end of the support channel is communicated with the corresponding inlet, and the other end defines an opening at the end of the support arm.

In some embodiments, the support channel has a first port close to the cabin body and a second port far away from the cabin body. In the direction from the first port to the second port, the inner diameter of the support channel is gradually reduced.

In some embodiments, the support arm includes a mounting seat, a connecting piece, and a support tube connected in sequence, where the mounting seat is connected to the cabin body, and the support tube is swingably connected to the mounting seat through the connecting piece.

In some embodiments, the cabin body includes an upper cover and a lower cover, which are enclosed to define the nail grinding chamber. The upper cover is transparent, and has a thickness that is gradually reduced in the direction from the center to the edge.

In some embodiments, the upper cover is snap-fitted with the lower cover.

In some embodiments, the upper cover and the lower cover are arranged in a hemispherical shape to form two halves of a sphere.

In some embodiments, the dust suction device includes a fan installation housing, a fan, and a motor. The fan installation housing defines a fan installation chamber, and an air inlet and air outlet communicating with the fan installation chamber, where the air inlet is communicated with the dust removal port. The fan is rotatably arranged in the fan installation chamber, and the motor is used to drive the fan.

In some embodiments, the dust suction cabin further includes a base, which cooperates with the fan installation housing to define a dust storage chamber. The dust storage chamber is communicated with the air outlet of the fan installation chamber. The nail grinding chamber, the fan installation chamber, and the dust storage chamber are arranged in order along the longitudinal direction.

In some embodiments, the inner diameter of the fan installation housing is gradually reduced along the direction from the air outlet to the air inlet.

In some embodiments, the dust suction cabin further includes a magnifying glass, which is movably arranged on the outside of the cabin body.

In some embodiments, the dust suction cabin further includes a light-emitting member, which is arranged on the chamber wall of the nail grinding chamber.

In some embodiments, the dust suction cabin further includes an aroma diffuser arranged on the outside of the cabin body, and a mist outlet of the aroma diffuser is in communication with the nail grinding chamber.

In some embodiments, the cabin body, the dust suction device, and the aroma diffuser are arranged in order along the vertical direction.

In some embodiments, the dust suction cabin further includes a PCB board arranged in the base, and a control program is pre-stored in the PCB board to control the startup and shutdown of the dust suction device.

In some embodiments, the dust suction cabin further includes a power supply arranged in the base and used to supply power to the PCB board, the dust suction device, the light emitting member, and the aroma diffuser.

The present disclosure also provides a nail grinder set, including a nail grinder and a dust suction cabin. The dust suction cabin includes a cabin body and a dust suction device. The cabin body defines a nail grinding chamber that is used for grinding the nail of a pet, and a chamber wall of the nail grinding chamber is at least partially transparent. The cabin body further defines a dust removal port and two inlets communicating with the nail grinding chamber. One of the two inlets is used for the nail grinder to enter the nail grinding chamber, and the other is used for the pet's claw to extend into the nail grinding chamber. The dust suction device is used for sucking out the scraps generated during the pet nail grinding process through the dust removal port.

The nail grinder includes a grinding piece, which can extend into the nail grinding chamber through the inlet of the dust suction cabin.

According to the dust suction cabin provided by the technical solution of the present disclosure, two support arms are installed at the two inlets of the cabin body, and the support channel of each support arm is communicated with the nail grinding chamber inside the cabin body, so the user can then move any one of the pet's four limbs together with the nail grinder to a relatively closed nail grinding chamber through the support channels of the two support arms to perform the nail grinding operation. At the same time, the pet's limbs or the nail grinder are wrapped by the inner peripheral wall of the corresponding support channel, which can effectively ensure the airtightness of the nail grinding chamber, and effectively avoid the situation that the waste debris from the nail grinding chamber is scattered to the outside of the cabin body. Then, the user can observe and move both the grinding piece of the nail grinder and the nail of the pet thus changing their positions through the transparent area in the wall of the nail grinding chamber to perform the nail grinding operation. Finally, the dust suction device is arranged at the dust removal port connected with the nail grinding chamber to suck off the waste debris produced during the pet nail grinding process. Thus, the waste debris produced during the pet nail grinding process can be discharged out of the nail grinding chamber in time, preventing the waste debris from floating in the nail grinding chamber or attaching to the pet's limbs and the nail grinder, there helping to improve the reliability of the dust suction cabin.

### BRIEF DESCRIPTION OF DRAWINGS

For a clearer understanding of the technical solutions that are used in the embodiments according to the present disclosure or that are used in the related art, hereinafter the drawings that are required for the description of the embodiments disclosed herein or the related art will be briefly introduced. Apparently, the drawings in the following description merely represent some embodiments of the present disclosure, and for those having ordinary skill in the art, other drawings may also be obtained based on the structures or arrangement shown in these drawings without investing creative efforts.

FIG. 1 is a schematic diagram illustrating a dust suction cabin according to an embodiment of the present disclosure.

FIG. 2 is an exploded schematic diagram of the dust suction cabin of FIG. 1.

FIG. 3 is a schematic diagram illustrating a partial structure of the dust suction cabin of FIG. 1.

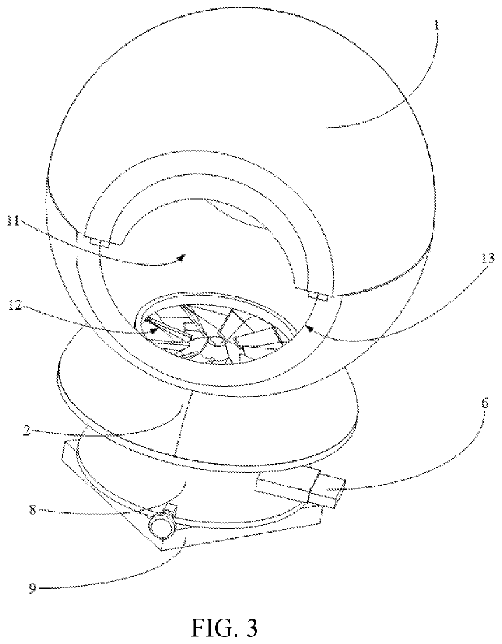

FIG. 4 is a schematic diagram illustrating a dust suction cabin according to another embodiment of the present disclosure.

FIG. 5 is a schematic diagram illustrating a dust suction cabin according to yet another embodiment of the present disclosure.

FIG. 6 is a schematic diagram illustrating a nail grinder set according to an embodiment of the present disclosure.

FIG. 7 is a schematic cross-sectional view of the nail grinder set of FIG. 6.

### DESCRIPTION OF REFERENCE SIGNS

1000. Dust suction cabin; 1. Cabin body; 11. Nail grinding chamber; 12. Dust removal port; 13. Inlet; 14. Upper cover; 15. Lower cover; 2. Dust suction device; 21. Fan installation housing; 211. Fan installation chamber; 212. Air inlet; 213. Air outlet; 22. Fan; 23. Motor; 3. Support arm; 31. Support channel; 32. Mounting seat; 33. Connecting piece; 34. Support tube; 4. Magnifying glass; 5. Light emitting member; 6. Aroma diffuser; 7. Base; 71. Dust storage chamber; 8. PCB board; 9. Power supply; 2000. Grinder; 200. Grinding piece

The realization of the objectives, functional features, and advantages of the present disclosure will be described in further detail in connection with the embodiments and with reference to the drawings.

### DETAILED DESCRIPTION OF ILLUSTRATIVE EMBODIMENTS

Hereinafter, technical solutions in the embodiments according to the present disclosure will be described in a definite and comprehensive manner in connection with the accompanying drawings in the embodiments according to the present disclosure. Apparently, the embodiments described herein are only a part, rather than all of the embodiments in accordance with the present disclosure. Based on the embodiments of the present disclosure, all other embodiments obtained by those having ordinary skill in the art without investing creative efforts shall all fall in the scope of protection of the present disclosure.

It should be noted that any directional or orientational indication (such as up, down, left, right, front, back . . . ) as used in the embodiments disclosed herein is merely used to explain the relative positional relationships and movement of the components in a specific posture (as shown in the figures). Should the particular posture changes, the directional or orientational indication would also change accordingly.

In addition, terms “first”, “second”, or the like as used herein are merely used for illustrative purposes, and shall not be construed as indicating relative importance or implicitly indicating the number of technical features specified. Thus, unless otherwise specified, the features defined by “first” and “second” may explicitly or implicitly include at least one of such feature. In addition, the technical solutions according to the various embodiments can be combined with each other, but it must be based on what can be achieved by a person having ordinary skill in the art. When the combination of technical solutions is conflicting or cannot be achieved, it should be considered that such a combination of technical solutions does not exist. nor does it fall in the scope of protection of the present disclosure.

The present disclosure provides a dust suction cabin 1000.

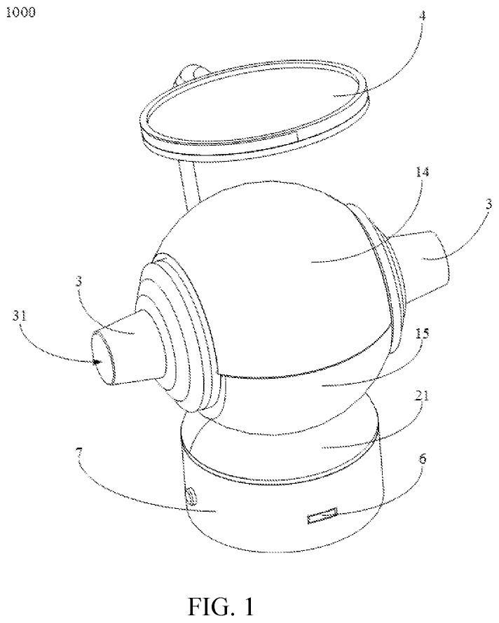

In an embodiment according to the disclosure, as illustrated in FIG. 1, the dust suction cabin 1000 includes a cabin body 1 and a dust suction device 2. The cabin body 1 defines a nail grinding chamber 11 that is used for grinding the nail of a pet, and a chamber wall of the nail grinding chamber 11 is at least partially transparent. The cabin body 1 further defines a dust removal port 12 and two inlets 13 communicating with the nail grinding chamber 11. One of the two inlets 13 is used for the nail grinder 2000 to enter the nail grinding chamber 11, and the other is used for the pet's claw to extend into the nail grinding chamber 11. The dust suction device 2 is used for sucking out the scraps generated during the pet nail grinding process through the dust removal port 12.

It is appreciated that according to the dust suction cabin 1000 provided by the technical solution of the present disclosure, two support arms 3 are installed at the two inlets 13 of the cabin body 1, and the support channel 31 of each support arm 3 is communicated with the nail grinding chamber 11 inside the cabin body 1, so the user can then move any one of the pet's four limbs together with the nail grinder 2000 to a relatively closed nail grinding chamber 11 through the support channels 31 of the two support arms 3 to perform the nail grinding operation. At the same time, the pet's limbs or the nail grinder 2000 are wrapped by the inner peripheral wall of the corresponding support channel 31, which can effectively ensure the airtightness of the nail grinding chamber 11, and effectively avoid the situation that the waste debris from the nail grinding chamber 11 is scattered to the outside of the cabin body 1. Then, the user can observe and move both the grinding piece 200 of the nail grinder 2000 and the nail of the pet thus changing their positions through the transparent area in the wall of the nail grinding chamber 11 to perform the nail grinding operation. Finally, the dust suction device 2 is arranged at the dust removal port 12 connected with the nail grinding chamber 11 to suck off the waste debris produced during the pet nail grinding process. Thus, the waste debris produced during the pet nail grinding process can be discharged out of the nail grinding chamber 11 in time, preventing the waste debris from floating in the nail grinding chamber 11 or attaching to the pet's limbs and the nail grinder 2000, there helping to improve the reliability of the dust suction cabin 1000.

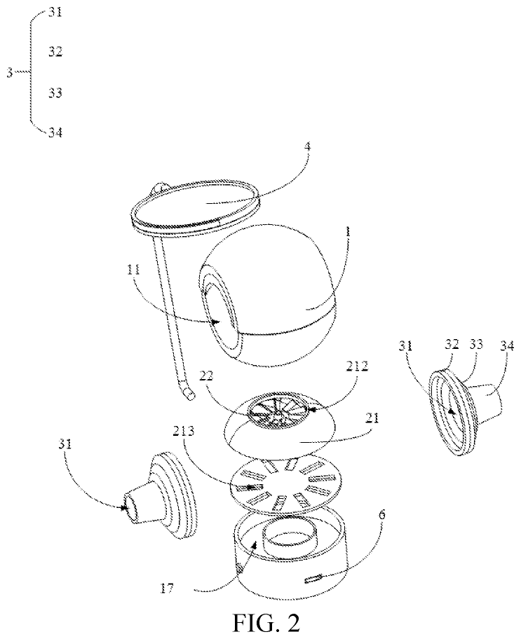

In some embodiments, as illustrated in FIG. 2, the two inlets 13 are respectively located on two opposite sides of the cabin body 1. It will be appreciated that by such an arrangement, after the nail grinder 2000 and the pet's limb separately extend into the nail grinding chamber 11 through the inlets 13, they would meet in the nail grinding chamber 11, thus facilitating the nail grinder 2000 to perform the nail grinding operation on the pet's nail. It is worth noting that the design of the present application will not be limited to this. In other embodiments, the two inlets 13 may be arranged in other manners, for example, but not limited to, the two inlets 13 are respectively located at two adjacent sides of the cabin body 1.

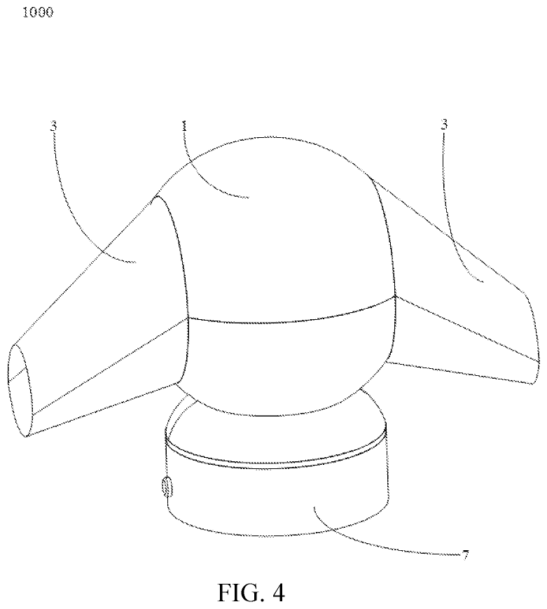

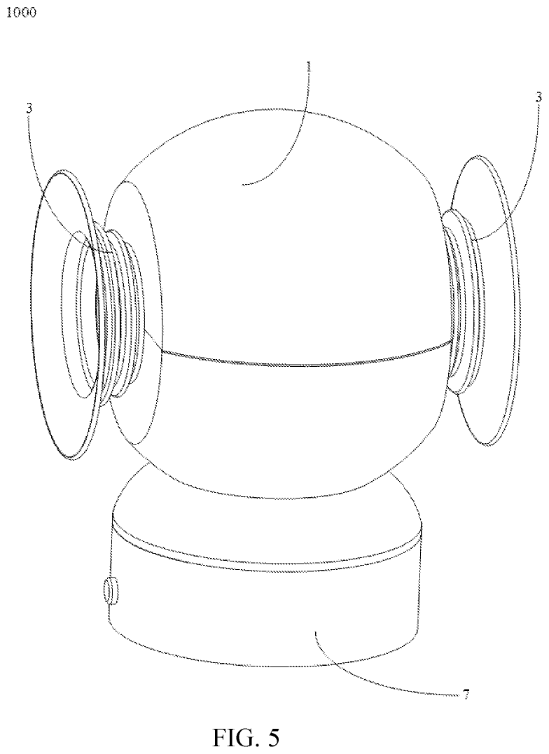

In some embodiments, as illustrated in FIG. 1, FIG. 4, or FIG. 5, the dust suction cabin 1000 further includes two support arms 3, which are respectively arranged at the two inlets 13 and extend from the inlets 13 in a direction away from the cabin body 1. The support arms 3 each define a support channel 31, where one end of the support channel 31 is communicated with the corresponding inlet 13, and the other end defines an opening at the end of the support arm 3. It can be understood that when the nail grinder 2000 and the pet's limb separately pass through the two support channels 31 and extend into the nail grinding chamber 11, the tube wall of the support channel 31 can support the nail grinder 2000 and the pet's limb respectively. At the same time, the support arms 3 also make it convenient for the user to hold the nail grinder 2000 or the leg of the pet that extends into the support channel 31. In addition, the support channels 31 extend in the direction away from the cabin body 1, which can also effectively prevent the debris produced during the pet nail grinding process from being scattered from the two inlets 13 in the cabin body 1 to the outside of the cabin body 1, thus improving the sealing performance of the dust suction cabin 1000 when in use.

Further, the support arm 3 is made of a tough or flexible material, such as but not limited to, plastic, silicone, or other materials.

In some embodiments, as illustrated in FIG. 4, the support channel 31 has a first port close to the cabin body 1 and a second port far away from the cabin body 1. In the direction from the first port to the second port, the inner diameter of the support channel 31 is gradually reduced. This arrangement helps to make the inner tube wall of the end of the support channel 31 away from the cabin body 1 fit the housing surface wall of the nail grinder 2000 or the skin of the pet's limb, thereby ensuring the airtightness of the nail grinding chamber 11.

In some embodiments, as illustrated in FIG. 2, the support arm 3 includes a mounting seat 32, a connecting piece 33, and a support tube 34 connected in sequence, where the mounting seat 32 is connected to the cabin body 1, and the support tube 34 is swingably connected to the mounting seat 32 through the connecting piece 33. It can be understood that the mounting seat 32 is used to be installed at the periphery of the inlet 13. The mounting base 32, the connecting piece 33 and the support tube 34 jointly form a support channel 31, and the support tube 34 is swingably connected to the mounting base 32 through the connecting piece 33. In this way, the user can swing the support tube 34 360° to adjust the extension direction of the support channel 31, which makes it convenient to adjust the position of the grinding piece 200 of the nail grinder 2000 or the nail of the pet in the nail grinding chamber 11 after the nail grinder 2000 or the limb of the pet passes through the support channel 31 into the nail grinding chamber 11. Specifically, the connecting piece 33 in the present application may be a corrugated tube.

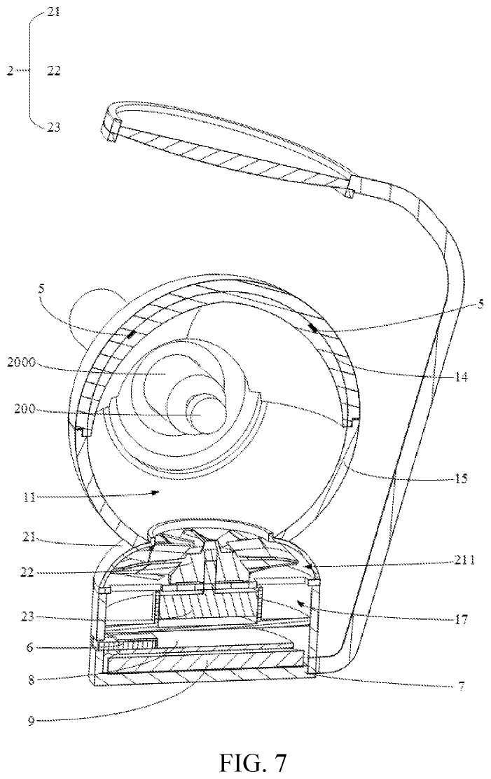

In some embodiments, as illustrated in FIG. 1 or 7, the cabin body 1 includes an upper cover 14 and a lower cover 15, which are enclosed to define the nail grinding chamber 11. The upper cover 14 is transparent, and has a thickness that is gradually reduced in the direction from the center to the edge. It can be understood that the thickness of the upper cover 14 made of a transparent material is tapered from the center to the edge. In other words, the upper cover 14 itself is the magnifying glass 4, so that the user can more clearly and carefully observe the internal conditions of the grinding chamber through the upper cover 14. It is worth noting that the design of the present application will not be limited to this. In other embodiments, the thickness of the upper cover 14 may not change from the center to the outer edge.

In some embodiments, as illustrated in FIG. 7, the upper cover 14 and the lower cover 15 are snap-fitted. Such an arrangement, on the one hand, is convenient for the user to remove the upper and lower cover 15 to clean the inner wall of the nail grinding chamber 11, and on the other hand, it is also convenient for the user to disassemble the cabin body 1 for storage and transportation. Further, the upper cover 14 and the lower cover 15 are arranged in a hemispherical shape, namely they are two halves of a complete sphere, which is not difficult to understand that on the one hand, it is convenient for the user to observe the nail grinding chamber 11 from all angles, and on the other hand, the inner surface of the chamber wall of the nail grinding chamber 11 being spherical is also helpful to prevent the residue of waste scraps produced during the pet nail grinding from sticking to the inner surface of the chamber wall of the nail grinding chamber 11.

In some embodiments, as illustrated in FIG. 7, the dust suction device 2 includes a fan installation housing 21, a fan 22, and a 23 motor. The fan installation housing 21 defines a fan installation chamber 211, and an air inlet 212 and air outlet 213 communicating with the fan installation chamber 211, where the air inlet 212 is communicated with the dust removal port 12. The fan 22 is rotatably arranged in the fan installation chamber 211, and the motor 23 is used to drive the fan 22. It can be understood that the air inlet 212 of the fan installation chamber 211 faces the air inlet end of the fan 22, and the air outlet 213 of the fan installation chamber 211 faces the air outlet end of the fan 22. The fan 22 is rotatably installed in the fan installation housing 21, and the motor 23 is drivingly connected to the fan 22. That is to say, the dust suction device 2 of the present application is independent of the cabin body 1, which can facilitate the maintenance and replacement of the fan installation housing 21 by the user.

In some embodiments, as illustrated in FIG. 2, the dust suction cabin 1000 further includes a base 7, which cooperates with the fan installation housing to define a dust storage chamber 71. The dust storage chamber 71 is communicated with the air outlet 213 of the fan installation chamber 211. The nail grinding chamber 11, the fan installation chamber 211, and the dust storage chamber 71 are arranged in order along the longitudinal direction. It can be understood that, according to the principle of gravity, such a configuration is beneficial for the waste scraps produced during the pet nail grinding process to move from the nail grinding chamber 11 to the dust storage chamber 71, thus improving the dust removal effect of the dust suction device 2 for the nail grinding chamber 11.

In some embodiments, as illustrated in FIG. 2 or FIG. 7, the inner diameter of the fan installation housing is tapered along the direction from the air outlet 213 to the air inlet 212. It can be understood that such a configuration on the one hand is conducive to reducing the diameter of the air inlet 212, thereby increasing the wind strength of the wind at the air inlet 212, and also facilitates the fan to exhaust the gas in the nail grinding chamber 11, so as to drive the waste scraps produced during the pet nail grinding process to move into the dust storage chamber 71. On the other hand, it is also conducive to reducing the volume required for the fan installation and reducing the manufacturing cost of the fan installation housing. In particular, the fan installation housing is installed at the bottom end of the lower cover 15, and is arranged in a hemispherical shape, and the outer surface of the fan installation housing is arranged opposite to the outer surface of the lower cover 15, which is beneficial to improve the look and feel of the product. The inner surface of the wall of the fan installation chamber is curved. As such, when the airflow flows on the curved surface, the speed of the airflow can be increased, which is beneficial to improve the dust suction efficiency of the dust suction device 2. It is worth noting that the design of the present application will not be limited to this. In other embodiments, the inner diameter of the fan installation housing may gradually expand or remain unchanged in the direction from the air outlet 213 to the air inlet 212.

In some embodiments, as illustrated in FIG. 1, the dust suction cabin 1000 further includes a magnifying glass 4, which is movably arranged on the outside of the cabin body 1. It can be understood that the dust suction cabin 1000 may further include a bracket movably arranged on the cabin body 1, and the magnifying glass 4 is installed on the bracket. Through the movable connection between the bracket and the cabin body 1, the position of the magnifying glass 4 can be adjusted to make it face the upper cover 14, which facilitates the user to observe the grinding conditions of pet nails inside the nail grinding chamber 11, which improves the safety of the product.

In some embodiments, the dust suction cabin 1000 may further include an LED lamp arranged at the magnifying glass 4, and the light of the LED lamp can overcome the situation that the user cannot clearly observe the nail grinding conditions inside the nail grinding chamber 11 due to the darkness of the environment.

In some embodiments, as illustrated in FIG. 7, the dust suction cabin 1000 may further include a light emitting member 5 that is provided on the chamber wall of the nail grinding chamber 11. It can be understood that, with this arrangement, the light emitting member 5 is used to emit visible light, thus making it convenient for the user to observe the nail grinding conditions of the pet in the nail grinding chamber 11, and at the same time, it also helps the pet owner to observe the blood lines on the pet's nail, thereby helping to prevent the nail grinder 2000 from accidentally hurting the blood-lined parts on the pet's nail, thereby improving the practicability and reliability of the dust suction cabin 1000. In particular, the light-emitting member 5 is an LED lamp.

In some embodiments, as illustrated in FIG. 1, the dust suction cabin 1000 may further include an aroma diffuser 6, which is arranged on the outer side of the cabin body 1, and a mist outlet of the aroma diffuser 6 is in communication with the nail grinding chamber 11. It can be understood that when the nail grinder 2000 is grinding the pet's nail in the nail grinding chamber 11, it is easy to emit unpleasant gas and remain inside the cabin body 1, so the aroma diffuser 6 can generate atomized gas with fragrance through ultrasonic waves. When the atomized gas with fragrance diffuses to various parts inside the cabin body 1, the peculiar smell inside the cabin body 1 can be effectively eliminated, thus improving the pet owner's experience of use.

In some embodiments, as illustrated in FIG. 7, the cabin body 1, the dust suction device 2, and the aroma diffuser 6 are arranged in order along the vertical direction. It can be understood that, according to the principle of gravity, such a configuration is beneficial for moving the waste debris in the nail grinding chamber 11 to the dust exhaust port and the air inlet of the dust suction device 2. In addition, the mist outlet of the aroma diffuser 6 is connected to the air outlet 213 of the dust suction device 2, and the motor 23 can drive the fan 22 to reverse, so that the atomized gas with fragrance can be delivered to the nail grinding chamber 11 to eliminate odors, which is simple and convenient.

In some embodiments, as shown in FIG. 7, the dust suction cabin 1000 further includes a PCB board 8 arranged in the base 7, and a control program is pre-stored in the PCB board 8 to control the startup and shutdown of the dust suction device 2. Correspondingly, the base 7 is provided with a control button electrically connected to the PCB board 8, and the user can enter a control instruction to the PCB board 8 through the control button.

In some embodiments, as illustrated in FIG. 7, the dust collection chamber 1000 further includes a power supply 9 arranged in the base 7, and the power supply 9 can be used to supply power to the PCB board 8, the dust collection device 2, the light emitting member 5, and the aroma diffuser 6.

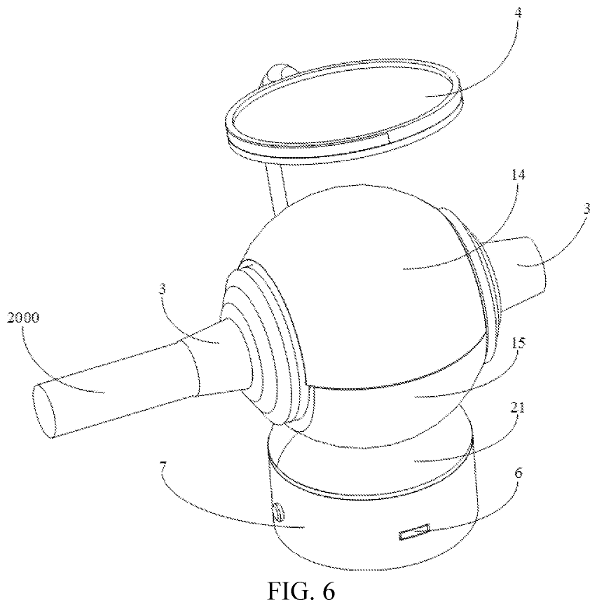

This disclosure also provides a nail grinder set. As illustrated in FIG. 6, the nail grinder set includes a nail grinder 2000 and a dust suction cabin 1000. The specific structure of the dust suction cabin 1000 refers to the above-mentioned embodiments. Since the nail grinder set adopts all the technical solutions of all the above embodiments, it has at least all the beneficial effects brought about by the technical solutions of the above-mentioned embodiments, which will not be repeated here. The nail grinder 2000 includes a grinding piece 200, which can extend into the nail grinding chamber 11 through the inlet 13 of the dust suction cabin 1000.

As illustrated in FIGS. 6 and 7, in particular, in some embodiments, the nail grinder 2000 is arranged in a cylindrical shape to fit the annular surface of the inner peripheral wall of the support channel 31. In other embodiments, the housing can also be arranged in other shapes, for example, but is not limited to, a rectangular shape.

The foregoing merely depicts some illustrative embodiments of the present disclosure, and is not intended to limit the scope of the present disclosure. Without departing from the inventive concept of the present disclosure, any equivalent structural changes made on the basis of the contents of the description and drawings of the present disclosure, or any direct/indirect use of the present disclosure on other related technical fields shall all be included in the scope of protection of the present disclosure.

What is claimed is:

1. A dust suction cabin used for grinding a pet nail, the dust suction cabin comprising:
a cabin body, which defines a nail grinding chamber used for grinding a nail of a pet, the nail grinding chamber comprising a chamber wall that is at least partially transparent; the cabin body further defines a dust removal port and two inlets that are in communication with the nail grinding chamber, wherein one of the two inlets is used for allowing a nail grinder to enter the nail grinding chamber, and the other is used for a claw of the pet to reach into the nail grinding chamber;
two support arms, which are respectively arranged at the two inlets and extend from the two inlets in a direction away from the cabin body, wherein the two support arms each define a support channel, wherein one end of the support channel is in communication with the corresponding inlet, and the other end defines an opening at the end of the support arm; and
a dust suction device, used for sucking out scraps produced during the pet nail grinding process through the dust removal port.

2. The dust suction cabin of claim 1, wherein the two support arms each comprise a mounting seat, a connecting piece, and a support tube that are connected in sequence, and wherein the mounting seat is connected to the cabin body, and the support tube is swingably connected to the mounting seat through the connecting piece.

3. The dust suction cabin of claim 1, wherein the cabin body comprises an upper cover and a lower cover, which are enclosed to define the nail grinding chamber, wherein the upper cover is transparent and has a thickness that is gradually reduced in the direction from the center to the edge.

4. The dust suction cabin of claim 2, wherein the cabin body comprises an upper cover and a lower cover, which are enclosed to define the nail grinding chamber, wherein the upper cover is transparent and has a thickness that is gradually reduced in the direction from the center to the edge.

5. The dust suction cabin of claim 1, wherein the dust suction device comprises a fan installation housing, a fan, and a motor; wherein the fan installation housing defines a fan installation chamber, and an air inlet and air outlet that are in communication with the fan installation chamber, wherein the air inlet is in communication with the dust removal port, and the fan is rotatably arranged in the fan installation chamber, and the motor is configured to drive the fan.

6. The dust suction cabin of claim 5, wherein an inner diameter of the fan installation housing is gradually reduced along the direction from the air outlet to the air inlet.

7. The dust suction cabin of claim 1, further comprising a magnifying glass movably arranged on an outside of the cabin body.

8. The dust suction cabin of claim 1, further comprising a light-emitting member arranged on the chamber wall of the nail grinding chamber.

9. The dust suction cabin of claim 1, further comprising an aroma diffuser arranged on the outside of the cabin body, the aroma diffuser comprising a mist outlet that is in communication with the nail grinding chamber.

10. The dust suction cabin of claim 9, wherein the cabin body, the dust suction device, and the aroma diffuser are arranged in order along a longitudinal direction.

11. A nail grinder set, comprising the dust suction cabin of claim 1, wherein nail grinder comprises a grinding piece operative to reach into the nail grinding chamber through the inlet of the dust suction cabin.

12. The nail grinder set of claim 11, wherein the two support arms each comprise a mounting seat, a connecting piece, and a support tube that are connected in sequence, and wherein the mounting seat is connected to the cabin body, and the support tube is swingably connected to the mounting seat through the connecting piece.

13. The nail grinder set of claim 11, wherein the cabin body comprises an upper cover and a lower cover, which are enclosed to define the nail grinding chamber, wherein the upper cover is transparent and has a thickness that is gradually reduced in the direction from the center to the edge.

14. The nail grinder set of claim 12, wherein the cabin body comprises an upper cover and a lower cover, which are enclosed to define the nail grinding chamber, wherein the upper cover is transparent and has a thickness that is gradually reduced in the direction from the center to the edge.

15. The nail grinder set of claim 11, wherein the dust suction device comprises a fan installation housing, a fan, and a motor; wherein the fan installation housing defines a fan installation chamber, and an air inlet and air outlet that are in communication with the fan installation chamber, wherein the air inlet is in communication with the dust removal port, and the fan is rotatably arranged in the fan installation chamber, and the motor is configured to drive the fan.

16. The nail grinder set of claim 15, wherein an inner diameter of the fan installation housing is gradually reduced along the direction from the air outlet to the air inlet.

17. The nail grinder set of claim 11, wherein the dust suction cabin further comprises a magnifying glass movably arranged on an outside of the cabin body.

18. The nail grinder set of claim 11, wherein the dust suction cabin further comprises a light-emitting member arranged on the chamber wall of the nail grinding chamber.

19. The nail grinder set of claim 11, wherein the dust suction cabin further comprises an aroma diffuser arranged on the outside of the cabin body, the aroma diffuser comprising a mist outlet that is in communication with the nail grinding chamber.

20. The nail grinder set of claim 19, wherein the cabin body, the dust suction device, and the aroma diffuser are arranged in order along a longitudinal direction.
